(19) United States
(12) Patent Application Publication
(10) Pub. No.: US 2021/0229092 A1
(43) Pub. Date: Jul. 29, 2021

(54) MICROFLUIDIC CHIP AND DETECTION METHOD USING MICROFLUIDIC CHIP

(71) Applicants: BEIJING BOE OPTOELECTRONICS TECHNOLOGY CO., LTD., Beijing (CN); BOE TECHNOLOGY GROUP CO., LTD., Beijing (CN)

(72) Inventors: Haochen CUI, Beijing (CN); Jian LI, Beijing (CN); Yingying ZHAO, Beijing (CN); Wenliang YAO, Beijing (CN); Peizhi CAI, Beijing (CN); Yue GENG, Beijing (CN); Le GU, Beijing (CN); Fengchun PANG, Beijing (CN); Yuelei XIAO, Beijing (CN); Hui LIAO, Beijing (CN); Nan ZHAO, Beijing (CN)

(21) Appl. No.: 16/644,904

(22) PCT Filed: Mar. 11, 2019
(86) PCT No.: PCT/CN2019/077687

§ 371 Date: Mar. 5, 2020

(51) Int. Cl. B01L 3/00; H01J 31/26; G01N 33/535; G01N 21/76

(52) U.S. Cl. CPC B01L 3/502715; H01J 31/26; B01L 2300/0645; G01N 21/76; G01N 33/535

(57) ABSTRACT

A microfluidic chip and a detection method using the microfluidic chip. The microfluidic chip includes: at least one micro-chamber; a photocathode located on a side of the at least one micro-chamber and configured to receive photons emitted from the micro-chamber to generate electrons; a micro-channel plate located on a side of the photocathode away from the micro-chamber and configured to multiply the electrons generated by the photocathode; and a first electrode located on a side of the micro-channel plate away from the photocathode; the micro-channel plate includes a plurality of micro-channels extending substantially in a thickness direction of the micro-channel plate, a secondary electron emission layer is provided on an inner wall of each of the plurality of micro-channels, and the first electrode is configured to detect the electrons that are multiplied by the micro-channel plate.

### TECHNICAL FIELD

Embodiments of the present disclosure provide a microfluidic chip and a detection method using microfluidic chip.

### BACKGROUND

A microfluidic chip is a device that can manipulate or detect fluids at a micrometer scale. Microfluidic chips have the ability to miniaturize the basic functions of laboratories such as biology and chemistry to a chip of a few square centimeters, so they are also called chip laboratories, Generally, a microfluidic chip includes micro-channels, and the micro-channels can form a network, so that the fluid can be controlled to flow in the network formed by the micro-channels, so as to achieve various functions of conventional chemical or biological laboratories. Therefore, the microfluidic chip has the advantages of small size, portability, flexible combination of functions, and high integration.

Biochemiluminescence (BCL) detection technology is a commonly used technology in biological and chemical detection. Biochemical luminescence detection technology is a quantitative analysis method based on a principle of a linear quantitative relationship between a concentration of an analyte in the chemical detection system and a chemiluminescence intensity of the system under certain conditions, a content of the analyte can be determined by detecting the chemiluminescence intensity of the system.

### SUMMARY

Embodiments of the present disclosure provides a microfluidic chip and a detection method using microfluidic chip. The microfluidic chip includes: at least one micro-chamber; a photocathode located on a side of the at least one micro-chamber and configured to receive photons emitted from the micro-chamber to generate electrons; a micro-channel plate located on a side of the photocathode away from the micro-chamber and configured to multiply the electrons generated by the photocathode; and a first electrode located on a side of the micro-channel plate away from the photocathode; the micro-channel plate includes a plurality of micro-channels extending substantially in a thickness direction of the micro-channel plate, a secondary electron emission layer is provided on an inner wall of each of the plurality of micro-channels, and the first electrode is configured to detect the electrons that are multiplied by the micro-channel plate. Therefore, the microfluidic chip can convert photons generated in the micro-chamber into electrons by the photocathode, and then the electrons are multiplied through the micro-channel plate, so that the optical signals generated by a biochemical luminescence reaction in the micro-chamber can be amplified to achieve qualitative or quantitative detection. In addition, because the micro-channel plate can be manufactured by using manufacturing apparatuses and processes of a liquid crystal display panel, with a low cost, thereby reducing the cost of the micro-fluidic chip.

At least one embodiment of the present disclosure provides a microfiuidic chip, which includes: at least one micro-chamber; a photocathode located on a side of the at least one micro-chamber and configured to receive photons emitted from the micro-chamber to generate electrons; a micro-channel plate located on a side of the photocathode away from the micro-chamber and configured to multiply the electrons generated by the photocathode; and a first electrode located on a side of the micro-channel plate away from the photocathode; the micro-channel plate includes a plurality of micro-channels extending substantially in a thickness direction of the micro-channel plate, a secondary electron emission layer is provided on an inner wall of each of the plurality of micro-channels, and the first electrode is configured to detect the electrons that are multiplied by the micro-channel plate.

For example, in the microfluidic chip provided by an embodiment of the present disclosure, each of the plurality of micro-channels has a cross-section with a size in a range from 20 to 40 microns, and has a length in a range from 0.6 to 2.4 millimeters.

For example, in the microfluidic chip provided by an embodiment of the present disclosure, a ratio of a length of each of the plurality of the micro-channels to a size of a cross-section of the micro-channel is in a range from 30 to 60.

For example, in the microfluidic chip provided by an embodiment of the present disclosure, the at least one micro-chamber includes a plurality of micro-chambers, the first electrode includes a plurality of first sub-electrodes, the plurality of the micro-chambers are provided in a one-to-one correspondence with the plurality of first sub-electrodes.

For example, in the microfluidic chip provided by an embodiment of the present disclosure, a ratio of a sum of volumes of the plurality of micro-channels to a volume of the micro-channel plate is in a range from 60% to 80%.

For example, in the microfluidic chip provided by an embodiment of the present disclosure, the plurality of the micro-channels are evenly distributed.

For example, in the microfluidic chip provided by an embodiment of the present disclosure, each of the micro-channels has a cross-section with a shape including at least one selected from the group consisting of a circle, a regular hexagon, and a regular octagon.

For example, in the microfluidic chip provided by an embodiment of the present disclosure, the microfluidic chip further includes: a second electrode located on a side of the micro-channel plate close to the photocathode, and the second electrode is configured to be loaded with a negative voltage.

For example, in the microfluidic chip provided by an embodiment of the present disclosure, a material of the photocathode includes at least one selected from the group consisting of gallium nitride, gallium arsenide, and indium gallium phosphide.

For example, in the microfluidic chip provided by an embodiment of the present disclosure, a material of the secondary electron emission layer includes alumina.

For example, in the microfluidic chip provided by an embodiment of the present disclosure, a material of the micro-channel plate includes glass.

At least one embodiment of the present disclosure provides a detection method using the microfluidic chip as described above, which includes: placing a detection reagent in the micro-chamber; modifying a substance to be detected by using a luminescent agent; introducing the substance to be detected modified with the luminescent agent into the micro-chamber to react with the detection reagent; introducing a luminescent substrate into the micro-chamber to cause the luminescent agent to emit light; and detecting, by the first electrode, the electrons multiplied by the micro-channel plate.

For example, in the detection method provided by an embodiment of the present disclosure, the detection reagent includes a capture antibody in an immune response, and the substance to be detected includes an antigen or an antibody corresponding to the capture antibody in a blood or urine sample.

For example, in the detection method provided by an embodiment of the present disclosure, the luminescent agent includes luminol.

For example, in the detection method provided by an embodiment of the present disclosure, the luminescent substrate includes horseradish peroxidase.

### BRIEF DESCRIPTION OF THE DRAWINGS

Hereinafter, the drawings accompanying embodiments of the present disclosure are simply introduced in order to more clearly explain technical solution(s) of the embodiments of the present disclosure. Obviously, the described drawings below are merely related to some of the embodiments of the present disclosure without constituting any, limitation thereto,

FIG. 1 is a schematic structural view illustrating a microfluidic chip provided by an embodiment of the present disclosure;

FIG. 2A is a schematic structural view illustrating another microfluidic chip provided by an embodiment of the present disclosure;

FIG. 2B is a schematic structural view illustrating another microfluidic chip provided by an embodiment of the present disclosure;

FIG. 3A is a schematic perspective view illustrating a micro-channel plate provided by an embodiment of the present disclosure;

FIG. 3B is a schematic plan view illustrating a micro-channel plate provided by an embodiment of the present disclosure;

FIG. 4 is a schematic structural view illustrating another microfluidic chip provided by an embodiment of the present disclosure; and

FIG. 5 is a flowchart illustrating a detection method using a microfluidic chip provided by an embodiment of the present disclosure.

### DETAILED DESCRIPTION

In order to make objectives, technical details and advantages of the embodiments of the present disclosure apparent, the technical solutions of the embodiments will be described in a clearly and fully understandable way in connection with the drawings related to the embodiments of the present disclosure. Apparently, the described embodiments are just a part but not all of the embodiments of the present disclosure. Based on the described embodiments herein, those skilled in the art can obtain other embodiment(s), without any inventive work, which should be within the scope of the present disclosure.

Unless otherwise defined, all the technical and scientific terms used herein have the same meanings as commonly understood by one of ordinary skill in the art to which the present disclosure belongs. The terms “first,” “second,” etc., which are used in the present disclosure, are not intended to indicate any sequence, amount or importance, but distinguish various components. Also, the terms “comprise,” “comprising,” “include,” “including,” etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly.

In the field of biochemiluminescence detection field, the most widely used is chemiluminescence immunoassay (CLIA) technology. Chemiluminescence immunoassay technology uses a chemiluminescence agent to directly label an antigen or an antibody. Because when an immune reaction occurs, the chemiluminescent substance is catalyzed by a catalyst and oxidized by an oxidant to form an intermediate in an excited state. When the intermediate in an excited state returns to a stable ground state, photons are emitted simultaneously. Chemiluminescence immunoassay technology can realize the quantitative analysis of immune response by detecting an amount of luminescence. However, in many application scenarios, because a light signal of the chemiluminescence immune response is weak and an amount of emitted light is small, it is necessary to use an expensive photomultiplier tube to amplify the light signal, which leads to high cost of the entire system using chemiluminescence immunoassay, and is not conducive to promotion and use.

Regarding to this, embodiments of the present disclosure provide a microfluidic chip and a detection method using the microfluidic chip. The microfluidic chip includes: at least one micro-chamber; a photocathode located on a side of the at least one micro-chamber and configured to receive photons emitted from the micro-chamber to generate electrons; a micro-channel plate located on a side of the photocathode away from the micro-chamber and configured to multiply the electrons generated by the photocathode; and a first electrode located on a side of the micro-channel plate away from the photocathode; the micro-channel plate includes a plurality of micro-channels extending substantially in a thickness direction of the micro-channel plate, a secondary electron emission layer is provided on an inner wall of each of the plurality of micro-channels, and the first electrode is configured to detect the electrons that are multiplied by the micro-channel plate. Therefore, the microfluidic chip can convert photons generated in the micro-chamber into electrons by the photocathode, and then the electrons are multiplied through the micro-channel plate, so that the optical signals generated by a biochemical luminescence reaction in the micro-chamber can be amplified to achieve qualitative or quantitative detection. In addition, because the micro-channel plate can be manufactured by using manufacturing apparatuses and processes of a liquid crystal display panel, with a low cost, thereby reducing the cost of the micro-fluidic chip.

Hereinafter, the microfluidic chip and the detection method using the microfluidic chip provided by the embodiments of the present disclosure will be described in detail with reference to the accompanying drawings.

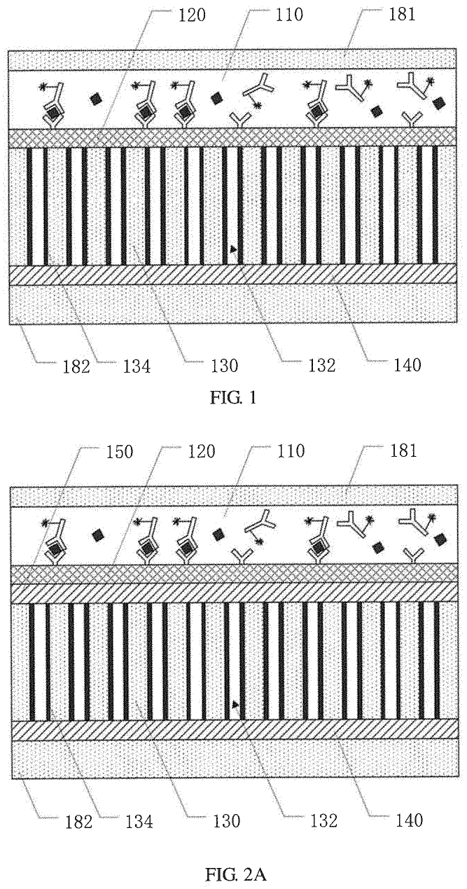

An embodiment of the present disclosure provides a microfluidic chip. FIG. 1 is a schematic structural view illustrating a microfluidic chip provided by an embodiment of the present disclosure. As illustrated in FIG. 1, the microfluidic chip includes at least one micro-chamber 110, a photocathode 120, a micro-channel plate 130, and a first electrode 140. The micro-chamber 110 can be used to perform a chemiluminescence reaction on a substance to be detected. The photocathode 120 is located on a side of the at least one micro-chamber 110 and is configured to receive photons emitted from the micro-chamber 110 to generate electrons. The micro-channel plate 130 is located on a side of the photocathode 120 away from the micro-chamber 110 and is configured to multiply the electrons generated by the photocathode 120. The first electrode 140 is located on a side of the micro-channel plate 130 away from the photocathode 120. The micro-channel plate 130 includes a plurality of micro-channels 132 extending substantially in a thickness direction of the micro-channel plate 130, each micro-channel 132 is provided with a secondary electron emission layer 134 at an inner wall of the channel 132. The first electrode 140 is configured to detect the electrons that are multiplied by the micro-channel plate 130. It should be explained that FIG. 1 is a schematic cross-sectional view taken along an extension direction of the micro-chamber, thus FIG. 1 only illustrates one micro-chamber 110. The embodiment of the present disclosure includes one micro-chamber but is not limited thereto, and the microfluidic chip may include a plurality of micro-chambers.

In the microfluidic chip provided by the embodiment of the present disclosure, a substance to be detected and a detection reagent may be placed in the at least one micro-chamber, and a luminescent agent may be added therein; when the substance to be detected reacts with the detection reagent, the luminescent agent upon being catalyzed or oxidized will emit photons; at this time, the photocathode can receive the photons emitted from the micro-chamber to generate electrons (photoelectrons); because the micro-channel plate includes a plurality of micro-channels extending substantially in a thickness direction of the micro-channel plate, and a secondary electron emission layer is provided on an inner wall of each micro-channel, and has a secondary emission coefficient greater than 1. After the electrons generated by the photocathode collide with the inner wall of the micro-channel, the number of secondary electrons emitted by the inner wall of the micro-channel is increased; After multiple collisions, the micro-channel plate can multiply the electrons generated by the photocathode, for example, 10⁵ times; then the first electrode can easily detect the electrons multiplied by the micro-channel plate. Therefore, each micro-channel can be regarded as an independent electron multiplier. The microfluidic chip can convert the photons generated in the micro-chamber into electrons by the photocathode, and then multiply the electrons by the micro-channel plate, thus a weak light signal generated by the biochemiluminescence reaction in the micro-chamber is amplified to realize the qualitative or quantitative detection of the substance to be detected. In addition, the microfluidic chip integrates a micro-channel plate and a micro-chamber for optical signal amplification, thereby improving the integration degree of the micro-fluidic, chip and increasing portability, thereby increasing the application scenario of the microfluidic chip. On the other hand, a plurality of micro-channels can be formed in a micro-channel plate by an etching process on a base (such as a glass base), and a secondary electron emission layer can be formed on an inner wall of the micro-channel by an atomic deposition process. The above-mentioned etching process and the atomic deposition process have relatively low cost, so that a fabrication cost of the micro-channel plate is relatively low. In addition, the micro-channel plate can also be manufactured by using manufacturing apparatuses and processes of a liquid crystal display panel, thereby further reducing the cost of the microfluidic chip. It should be explained that, in order to better explain the microfluidic chip provided by this embodiment, this embodiment describes an exemplary process of reacting and generating photons in a micro-chamber; however, the embodiments of the present disclosure include, but are not limited thereto, the way of generating photons in a micro-chamber can also be of other types.

For example, as illustrated in FIG. 1, the microfluidic chip further includes an upper cover plate 181 located on a side of the micro-chamber 110 away from the photocathode 120 and a lower cover plate 182 located on a side of the first electrode 140 away from the micro-channel plate 130, to protect the microfluidic chip.

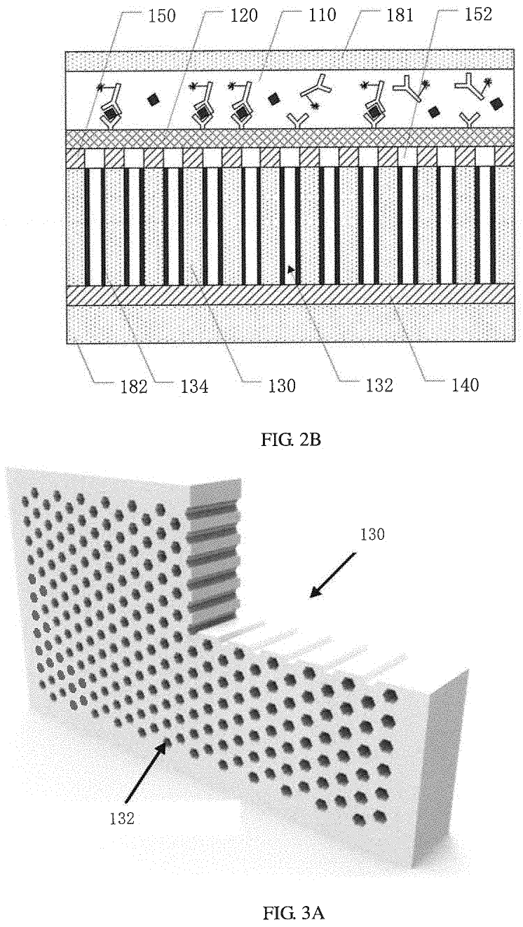

FIG. 2A is a schematic structural view illustrating another microfluidic chip provided by an embodiment of the present disclosure; and FIG. 2B is a schematic structural view illustrating another microfluidic chip provided by an embodiment of the present disclosure. As illustrated in FIG. 2A and FIG. 2B, the microfluidic chip further includes a second electrode 150, which is located on a side of the micro-channel plate 130 close to the photocathode 120, and is configured to be loaded with a negative voltage. The second electrode 150 can form a negative electric field, that is, a direction of the electric field is from the micro-channel plate 130 to the micro-chamber 110, so that the electrons generated by the photocathode 120 can enter the micro-channel 132 of the micro-channel plate 130 at a certain angle and with a high speed, and hit the secondary electron emission layer 134 on the inner wall of the micro-channel 132 to cause the micro-channel plate to multiply the electrons generated by the photocathode.

For example, as illustrated in FIG. 2A, the second electrode 150 may be a continuous electrode. As illustrated in FIG. 2B, the second electrode 150 may also be an electrode including a plurality of via holes 152, and the plurality of via holes 152 are arranged in a one-to-one correspondence with the plurality of micro-channels 132 in the micro-channel plate 130, that is, an orthographic projection of the plurality of via holes 152 on the micro-channel plate 130 overlaps with a plurality of micro-channels 132, thereby reducing an obstruction of electrons under the premise of generating an electric field.

For example, a material of the second electrode may be indium tin oxide (ITO). Of course, the embodiments of the present disclosure include but are not limited thereto.

For example, in some exemplary embodiments, a material of the photocathode includes gallium nitride, gallium arsenide, or indium gallium phosphide. Of course, the embodiments of the present disclosure include but are not limited thereto.

For example, in some exemplary embodiments, a material of the secondary electron emission layer includes alumina. Of course, the embodiments of the present disclosure include but are not limited thereto.

For example, in some exemplary embodiments, a material of the micro-channel plate includes glass, which can reduce a cost of the micro-channel plate. Of course, the embodiments of the present disclosure include, but are not limited thereto, the material of the micro-channel plate may also be alumina ceramics.

For example, in some exemplary embodiments, the number of micro-channels in a micro-channel plate is in a range from 10⁵ to 10⁶ such as 10⁶.

For example, in a microfluidic chip provided by an embodiment of the present disclosure, a ratio of a length of each micro-channel to a size of a cross-section of each micro-channel is in a range from 30 to 60. Because the length of the micro-channel is much larger than the size of the cross-section of the micro-channel, the electrons generated by the photocathode will collide back and forth between the inner walls of the micro-channel, thereby multiplying multiple times, and causing the electrons generated by the photocathode of the micro-channel plate to be multiplied to 10⁴-10⁶ times. It should be explained that the size of the cross-section of the micro-channel refers to a maximum size of the cross-section of the micro-channel. For example, in the case where the cross-section of the micro-channel is circular, the size of the cross-section of the micro-channel is a diameter of the circle; and in the case where the cross-section of the micro-channel is a regular polygon, the size of the cross-section of the micro-channel is a diameter of a circumscribed circle of the regular polygon.

For example, in some exemplary embodiments, the size of the cross-section of each micro-channel is in a range from 20 to 40 microns, and a length of each micro-channel is in a range from 0.6 to 2.4 mm, thereby causing the micro-channel plate to multiply the electrons generated by the photocathode by 10⁴-10⁶ times. In addition, the size of the cross-section of the micro-chamber is compatible with the manufacturing apparatus and process of the liquid crystal display panel, which facilitates reducing the manufacturing cost.

For example, m some exemplary embodiments, a ratio of a sum of volumes of the plurality of the micro-channels to a volume of the micro-channel plate is in a range from 60% to 80%, that is, on a surface of the micro-channel plate, a ratio of a sum of areas occupied by the plurality of micro-channels and an area of the surface of the micro-channel plate is in a range from 60% to 80%. On one hand, the micro-channel plate can have a certain strength, and on the other hand, the micro-channel plate can have a high multiplication effect on electrons.

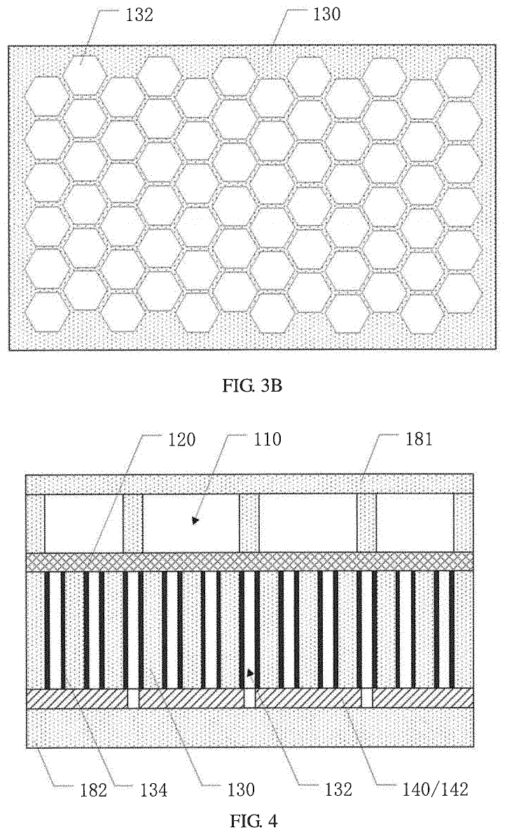

FIG. 3A is a schematic perspective view illustrating a micro-channel plate provided by an embodiment of the present disclosure; and FIG. 3B is a schematic plan view illustrating a micro-channel plate provided by an embodiment of the present disclosure. As illustrated in FIG. 3A and FIG. 3B, the plurality of micro-channels 132 are evenly distributed, thereby causing a consistency of the multiplication effect of the entire micro-channel plate 130 on electrons. In the case where the mierofluidic chip has a plurality of micro-chambers, the micro-channel plate has the same multiplication effect on the optical signals generated in different micro-chambers.

For example, in some exemplary embodiments, a shape of a cross-section of each micro-channel includes a circle, a regular hexagon, or a regular octagon. As illustrated in FIG. 3B, in the case where the shape of the cross-section of each micro-channel is a regular hexagon, the arrangement of the plurality of micro-channels can be made closer, and the ratio of the sum of the volumes of the plurality of micro-channels to the volume of the micro-channel plate is higher.

For example, in some exemplary embodiments, in a micro-channel plate, an angle formed between an extension direction of the micro-channel and a normal line of the micro-channel plate is less than 8 degrees.

FIG. 4 is a schematic plan view illustrating a microfluidic chip provided by an embodiment of the present disclosure. As illustrated in FIG. 4, the microfluidic chip includes a plurality of micro-chambers 110, that is, the at least one micro-chamber 110 includes a plurality of micro-chambers 110. FIG. 4 illustrates four micro-chambers 110, however, the number of micro-chambers in the embodiment of the present disclosure includes but is not limited to four. The first electrode 140 includes a plurality of first sub-electrodes 142, and the plurality of micro-chambers 110 are arranged in a one-to-one correspondence with the plurality of first sub-electrodes 142. Orthographic projections of the plurality of micro-chambers 110 on the lower cover plate 182 fall into orthographic projections of the plurality of first sub-electrodes 142 on the lower cover plate 182, respectively. In this case, the plurality of first sub-electrodes 142 can detect the optical signals generated by the plurality of micro-chambers 110, respectively. Therefore, the microfluidic chip can perform multiple detection functions simultaneously.

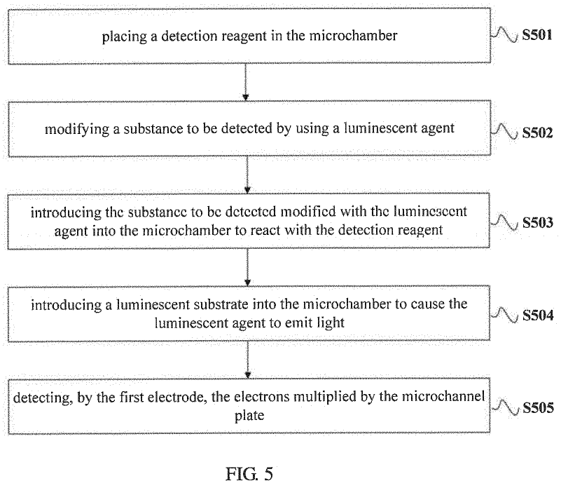

An embodiment of the present disclosure further provides a detection method using the above microfluidic chip. FIG. 5 is a flowchart illustrating a detection method using a microfluidic chip provided by an embodiment of the present disclosure. As illustrated in FIG. 5, the detection method includes the following steps S501-S505.

Step S501: placing a detection reagent in the micro-chamber.

Step S502: modifying a substance to be detected by using a luminescent agent.

Step S503: introducing the substance to be detected modified with the luminescent agent into the micro-chamber to react with the detection reagent.

Step S504: introducing a luminescent substrate into the micro-chamber to cause the luminescent agent to emit light.

Step S505: detecting, by the first electrode, the electrons multiplied by the micro-channel plate.

In the detection method using a microfluidic chip provided by the embodiment of the present disclosure, in a micro-chamber, a substance to be detected modified with a luminescent agent reacts with a detection reagent, and the luminescent substrate causes the luminescent agent to emit light (for example, causing the luminescent agent to emit light by catalysis and oxidation reactions). At this time, the photocathode can receive the photons emitted from the micro-chamber to generate electrons; because the micro-channel plate includes a plurality of micro-channels extending substantially in a thickness direction of the micro-channel plate, and an inner wall of each micro-channel is provided with a secondary having a secondary emission coefficient greater than 1. Upon the electrons generated by the photocathode colliding with the inner wall of the micro-channel, the number of secondary electrons emitted from the inner wall of the micro-channel is increased. After multiple collisions, the electrons generated by the photocathode can be multiplied by the micro-channel plate, for example, by 10⁵ times; then the electrons multiplied by the micro-channel plate can be easily detected by the first electrode. Therefore, the detection method can use the microfluidic chip to amplify a weak light signal generated by the biochemical luminescence reaction in the micro-chamber, so as to realize the qualitative or quantitative detection of the substance to be detected.

For example, in some exemplary embodiments, the above-mentioned detection reagent includes a capture antibody in an immune response, and the substance to be detected includes an antigen or antibody corresponding to the capture antibody in a blood or urine sample. For example, the above-mentioned detection reagent includes bovine globulin G, and the substance to be detected includes goat anti-bovine immunoglobulin G. Of course, the embodiments of the present disclosure include but are not limited thereto.

For example, in some exemplary embodiments, the luminescent agent includes luminol.

For example, in some exemplary embodiments, the luminescent substrate includes horseradish peroxidase.

An embodiment of the present disclosure further provides a manufacturing method of a microfluidic chip, which includes: forming at least one micro-chamber; forming a photocathode located on a side of the at least one micro-chamber, the photocathode being configured to receive photons emitted from the micro-chamber to generate electrons; forming a micro-channel plate located on a side of the photocathode away from the micro-chamber, the micro-channel plate being configured to multiply the electrons generated by the photocathode; and forming a first electrode located on a side of the micro-channel plate away from the photocathode, the micro-channel plate including a plurality of micro-channels extending substantially in a thickness direction of the micro-channel plate; each of the plurality of micro-channels is provided with a secondary electron emission layer at an inner wall of the micro-channel, and the first electrode being configured to detect the electrons multiplied by the micro-channel plate.

For example, in some exemplary embodiments, the manufacturing method further includes: forming the micro-channel plate located on a side of the photocathode away from the micro-chamber includes: preparing the micro-channel plate; and forming the micro-channel plate located on a side of the photocathode away from the micro-chamber.

For example, in some exemplary embodiments, preparing the micro-channel plate includes: providing a glass base plate; forming a plurality of micro-channels in the glass base plate by an etching process; and forming a secondary electron emission layer on an inner wall of each of the plurality of micro-channels by an atomic deposition process. In this case, a micro-channel plate can form a plurality of micro-channels by an etching process on a base plate (such as a glass base plate), and form a secondary electron emission layer on an inner wall of the micro-channels by an atomic deposition process. The above-mentioned etching process and atoms deposition process have relatively low cost, so the fabrication cost of the micro-channel plate is relatively low. In addition, the micro-channel plate can also be manufactured by using manufacturing apparatuses and processes of a liquid crystal display panel, thereby further reducing the cost of the microfluidic chip.

For example, in some exemplary embodiments, forming a plurality of micro-channels on a glass base plate by arm etching process includes: coating photoresist on the glass base plate; exposing the photoresist by using an exposure machine to form a photoresist pattern including a plurality of via holes; using the photoresist pattern as a mask to etch the glass base plate to form a plurality of micro-channels therein; and removing the photoresist pattern.

The following should be explained.

(1) The accompanying drawings involve only the structure(s) in connection with the embodiment(s) of the present disclosure, and other structure(s) may be referred to common design(s).

(2) In case of no conflict, features in one embodiment or in different embodiments may be combined.

The above are only specific implementations of the present disclosure, the protection scope of the present disclosure is not limited thereto. Any changes or substitutions easily occur to those skilled in the art within the technical scope of the present disclosure should be covered in the protection scope of the present disclosure. Therefore, the protection scope of the present disclosure should be based on the protection scope of the claims.

1. A microfluidic chip, comprising:
at least one micro-chamber;
a photocathode, located on a side of the at least one micro-chamber and configured to receive photons emitted from the micro-chamber to generate electrons;
a micro-channel plate located on a side of the photocathode away from the micro-chamber and configured to multiply the electrons generated by the photocathode; and
a first electrode located on a side of the micro-channel plate away from the photocathode,
wherein the micro-channel plate comprises a plurality of micro-channels extending substantially in a thickness direction of the micro-channel plate, a secondary electron emission layer is provided on an inner wall of each of the plurality of micro-channels, and the first electrode is configured to detect the electrons that are multiplied by the micro-channel plate.

2. The microfluidic chip according to claim 1, wherein each of the plurality of micro-channels has a cross-section with a size in a range from 20 to 40 microns, and has a length in a range from 0.6 to 2.4 millimeters.

3. The microfluidic chip according to claim 1, wherein a ratio of a length of each of the plurality of the micro-channels to a size of a cross-section of the micro-channel is in a range from 30 to 60.

4. The microfluidic chip according to claim 1, wherein the at least one micro-chamber comprises a plurality of micro-chambers, the first electrode comprises a plurality of first sub-electrodes, the plurality of the micro-chambers are provided in a one-to-one correspondence with the plurality of first sub-electrodes.

5. The microfluidic chip according to claim 1, wherein a ratio of a sum of volumes of the plurality of micro-channels to a volume of the micro-channel plate is in a range from 60% to 80%.

6. The microfluidic chip according to claim 1, wherein the plurality of the micro-channels are evenly distributed.

7. The microfluidic chip according to claim 1, wherein each of the micro-channels has a cross-section with a shape comprising at least one selected from the group consisting of a circle, a regular hexagon, and a regular octagon.

8. The microfluidic chip according to claim 1, further comprising:
a second electrode located on a side of the micro-channel plate close to the photocathode,
wherein the second electrode is configured to be loaded with a negative voltage.

9. The microfluidic chip according to claim 1, wherein a material of the photocathode comprises at least one selected from the group consisting of gallium nitride, gallium arsenide, and indium gallium phosphide.

10. The microfluidic chip according to claim 1, wherein a material of the secondary electron emission layer comprises alumina.

11. The microfluidic chip according to claim 1, wherein a material of the micro-channel plate comprises glass.

12. A detection method using the microfluidic chip according to claim 1, comprising:
placing a detection reagent in the micro-chamber;
modifying a substance to be detected by using a luminescent agent;
introducing the substance to be detected modified with the luminescent agent into the micro-chamber to react with the detection reagent;
introducing a luminescent substrate into the micro-chamber to cause the luminescent agent to emit light; and
detecting, by the first electrode, the electrons multiplied by the micro-channel plate.

13. The detection method according to claim 12, wherein the detection reagent comprises a capture antibody in an immune response, and the substance to be detected comprises an antigen or an antibody corresponding to the capture antibody in a blood or urine sample.

14. The detection method according to claim 12, wherein the luminescent agent comprises luminol.

15. The detection method according to claim 12, wherein the luminescent substrate comprises horseradish peroxidase.

16. The microfluidic chip according to claim 2, wherein the at least one micro-chamber comprises a plurality of micro-chambers, the first electrode comprises a plurality of first sub-electrodes, the plurality of the micro-chambers are provided in a one-to-one correspondence with the plurality of first sub-electrodes.

17. The microfluidic chip according to claim 2, wherein a ratio of a sum of volumes of the plurality of micro-channels to a volume of the micro-channel plate is in a range from 60% to 80%.

18. The microfluidic chip according to claim 2, further comprising:
a second electrode located on a side of the micro-channel plate close to the photocathode,
wherein the second electrode is configured to be loaded with a negative voltage.

19. The microfluidic chip according to claim 2, wherein a material of the photocathode comprises at least one selected from the group consisting of gallium nitride, gallium arsenide, and indium gallium phosphide.
